(19) United States
(12) Patent Application Publication
(10) Pub. No.: US 2007/0298149 A1
(43) Pub. Date: Dec. 27, 2007

(54) Animal Feedstuff Dyeing and Animal Drugs

(76) Inventor: Ralf Schweinfurth, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/665,749

(22) PCT Filed: Oct. 18, 2005
(86) PCT No.: PCT/EP05/11207

§ 371 Date: Apr. 19, 2007

(30) Foreign Application Priority Data
Oct. 20, 2004 (DE) 102004051244.2

(51) Int. Cl. A23K 1/16; A61K 47/02; A61K 47/48; A61K 9/28; C09C 1/00; C09C 1/24

(52) U.S. Cl. 426/250

(57) ABSTRACT

The invention relates to the use of effect-producing pigments based on plate-shaped substrates for dyeing animal feedstuffs and animal drugs.

The present invention relates to the use of effect pigments based on flake-form substrates for colouring feed products and animal medicaments.

Besides functional applications, effect pigments are also increasingly being employed for the visual upgrading of products. The use of pearlescent or interference pigments for colouring products from the foods and pharmaceuticals sectors is known from WO 00/03609 and WO 03/063616. The use of TiO₂/mica pigments in fish food is disclosed in WO 98/56261. It should also be possible to employ effect pigments in the animal feeds sector for improving the visualisation and differentiation of feed products so long as the pigments are prepared under the corresponding strict purity and quality regulations.

The object of the present invention is to increase the amount of feed by increasing the visual attractiveness of the feed to the animals. Improved feed uptake results in improved feed utilisation by the animals. This also significantly reduces the amount of unutilised feed product residues. Such feed product residues can have an adverse effect in the form of increased microorganism and residue levels in the feed area or water. They are also associated with increased costs for cleaning and waste-water treatment.

Surprisingly, it has now been found that the uptake of amount of feed in animals can be increased if effect pigments are admixed with the feed products. The increase in the visual attractiveness of the feed products furthermore enables easier recognition of the feed by the animals. A further advantage is that the colouring enables better visualisation and differentiation of feed products with different active-ingredient additives (for example vitamins, enzymes, trace elements, essential fats and/or minerals) by the animal keeper. This reduces the risks emanating from mistaken identification or overdosing of the individual feed products. This method is likewise very highly suitable for the visual differentiation and identification of animal medicaments.

This increase in the visual attractiveness by effect pigments can also successfully be used for so-called attractant and bait feed products, such as, for example, angling bait, since the animal perceives the attractant feed coloured in this way or the bait better.

The present invention thus relates to the use of effect pigments in feed products and animal medicaments.

All feed products and animal medicaments suitable for colouring can be optically upgraded using effect pigments. The feed products can be in all forms of administration, such as, for example, powders, grains, pellets, extrudates, compressed products, expanded products, granules, in liquid form, etc. Preferred feed products are those which are administered orally.

The coloured animal medicaments can be administered either as a powder or in compact form, for example tablets, dragees, gelatin capsules, suppositories, or in liquid form, for example cough mixture.

The feed products to be coloured may furthermore originate from all feed areas, such as, for example, fish food, pet food, fattening feed, concentrated feed, bird and poultry feed, dog and cat food, bait and attractant feed, etc. The feed products to be coloured can be of either animal or vegetable origin. By means of a suitable coating composition, grains, seed or other grown feed products, for example, can be coloured on the surface with effect pigments.

Suitable effect pigments are, in particular, those which are based on flake-form substrates covered with one or more layers of metal oxides. Particularly preferred metal oxides are TiO₂, Fe₂O₃ and Fe₃O₄, furthermore BiOCl, SiO₂ and Al₂O₃. In particular, the combination of effect pigments with TiO₂ and/or Fe₃O₄ layers with natural or nature-identical dyes, coloured pigments or colouring fruit and plant extracts, FD&C and D&C colorants help the feed products to achieve increased visual attractiveness to the animals.

In contrast to coloured pigments approved in the animal feeds sector, for example pure titanium dioxide E171, the effect pigments based on flake-form substrates can be dispersed very easily in the medium to be pigmented. Furthermore, the feed products coloured in this way are distinguished by increased light and moisture protection. In particular, vitamin preparations have a longer life.

It is found that the feed products and animal medicaments can be provided with novel interesting hues and at the same time novel properties even with very small amounts of effect pigments. Excellent results are achieved on colouring of the product with only 0.01 to 50.0% by weight, preferably 0.05 to 40% by weight, in particular 0.1 to 30% by weight, of effect pigment, based on the feed product or animal medicament.

In the case that the effect pigment is admixed directly with the feed product or animal medicament during manufacture, the amount of effect pigment used is preferably 0.5 to 30% by weight, in particular 1 to 20% by weight and very particularly preferably 3 to 10% by weight. In the case of the surface treatment of feed products, the use range is 0.1 to 25% by weight, preferably 0.5 to 20% by weight, in particular 1 to 15% by weight, based on the colouring or coating solution. It is also possible to add a mixture of different effect pigments to the feed product or animal medicament.

Suitable effect pigments are both pigments which are covered with only one metal-oxide layer and also pigments whose substrates have several (two or more) layers

Suitable base substrates for the effect pigments according to the invention are transparent or semi-transparent flake-form substrates. Preferred substrates are phyllosilicates. Particularly suitable are natural and/or synthetic mica, talc, kaolin, flake-form iron or aluminium oxides, glass flakes, SiO₂ flakes, TiO₂ flakes, flake-form mixed oxides, such as, for example, FeTiO₃, Fe₂TiO₅, graphite flakes or other comparable materials, depending on the respective legal permissibility for use in animal feed or in animal medicaments.

not crucial per se and can be matched to the particular application. In general, relatively large substrates give rise to an improved sparkle effect and consequently better visualisation for the animals to be fed. In general, the flake-form substrates have a thickness of between 0.005 and 10 μm, in particular between 0.05 and 5 μm. The dimension in the two other ranges is usually between 1 and 600 μm, preferably between 2 and 400 μm, and in particular between 5 and 150 μm.

In the case of covering of the base substrates with only one metal-oxide layer, the thickness of the layer is preferably 5-500 nm, in particular 20-450 nm and very particularly preferably 40-350 nm. In the case of multilayered pigments, which preferably contain two, three, five or seven layers, the total layer thickness should not exceed 1000 nm.

Suitable effect pigments having one layer are preferably mica flakes, glass flakes, Al₂O₃ flakes, which are coated with TiO₂, Fe₂O₃, Fe₃O₄ or a mixture of TiO₂/Fe₂O₃. Particular preference is given to effect pigments based on mica which are coated with TiO₂, Fe₂O₃ or Fe₃O₄. The TiO₂ can be in either the anatase or the rutile modification.

Suitable multilayered pigments are, in particular, pigments based on multicoated substrates, which are frequently distinguished by the fact that they have an alternating high- and low-refractive-index metal-oxide layer.

Particularly suitable are multilayered pigments based on multicoated flake-form substrates which have at least one layer sequence (A) (B) (A), where
(A) is a high-refractive-index coating consisting of titanium dioxide and/or iron oxide, preferably Fe₂O₃, and
(B) is a low-refractive-index coating having a refractive index n≦1.8, preferably comprising SiO₂ and/or Al₂O₃.

The thickness of the individual layers (A) and (B) of high or low refractive index respectively on the base substrate is crucial for the optical properties of the pigment. For the multilayered pigment having an intense lustre effect, the thickness of the individual layers must be set precisely to one another.

The thickness of layer (A) is generally 10-500 nm, preferably 20-400 nm, in particular 30-350 nm. The thickness of layer (B) is generally 10-500 nm, preferably 20-400 nm, in particular 30-350 nm.

The pigments may comprise a plurality of identical or different combinations of layer packages, but the substrate is preferably covered with only one layer package (A) (B) (A). In order to intensify the tinting strength, the pigment according to the invention can comprise up to 4 layer packages, but where the thickness of all layers on the substrate should not exceed 3 μm. An odd number of layers is preferably applied to the flake-form substrate, with a high-refractive-index layer as both the innermost and outermost layer. Particular preference is given to a construction of three optical interference layers in the sequence (A) (B) (A). A suitable high-refractive-index layer is preferably a layer of TiO₂, Fe₂O₃ and/or Fe₃O₄. The TiO₂ here can be in the rutile or anatase modification.

Colourless low-refractive-index materials which are suitable for the coating (B) are preferably metal oxides or the corresponding oxide hydrates, such as, for example, SiO₂, Al₂O₃, AlO(OH), B₂O₃, MgF₂, MgSiO₃ or a mixture of the said metal oxides, corresponding to the legal approvals for use in feed products and animal medicaments. Layer (B) is, in particular, an SiO₂ layer.

The known multilayered pigments can be prepared relatively easily by producing a plurality of high- and low-refractive-index interference layers of precisely defined thickness and smooth surface on the finely divided flake-form substrates.

The metal-oxide layers are preferably applied to the substrate to be coated by wet-chemical methods, it being possible to use the wet-chemical coating methods developed for the preparation of pearlescent pigments. Methods of this type are described, for example, in DE 14 67 468, DE 19 59 988, DE 20 09 566, DE 22 14545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017 or also in further patent documents and other publications known to the person skilled in the art.

Furthermore, the coating can also be carried out in a fluidised-bed reactor by gas-phase coating, it being possible to use correspondingly, for example, the processes proposed in EP 0 045 851 and EP 0 106 235 for the preparation of pearlescent pigments.

The hue of the effect pigments can be varied in very broad limits through the different choice of the covering amounts or the layer thicknesses resulting therefrom. The fine tuning for a certain hue can be achieved beyond the pure choice of amount by approaching the desired colour under visual or measurement-technology control.

Particularly preferred effect pigments for feed products or animal medicaments have the following layer structure:

substrate + TiO₂
substrate + Fe₂O₃
substrate + Fe₃O₄
substrate + TiO₂ + SiO₂ + TiO₂
substrate + TiO₂ + SiO₂ + Fe₂O₃
substrate + TiO₂ + SiO₂ + Fe₃O₄
substrate + TiO₂ + Al₂O₃ + TiO₂
substrate + TiO₂ + Al₂O₃ + Fe₂O₃
substrate + TiO₂ + Al₂O₃ + Fe₂O₃
substrate + Fe₂O₃ + SiO₂ + TiO₂
substrate + Fe₃O₄ + SiO₂ + TiO₂
substrate + Fe₂O₃ + Al₂O₃ + TiO₂
substrate + Fe₃O₄ + Al₂O₃ + TiO₂
substrate + SiO₂ + TiO₂
substrate + SiO₂ + Fe₂O₃
substrate + SiO₂ + Fe₃O₄

In particular, the above-mentioned effect pigments are based on mica flakes, furthermore on glass flakes, SiO₂ flakes or Al₂O₃ flakes.

The effect pigments used are preferably Fe₂O₃—, magnetite- and/or TiO₂-coated natural or synthetic mica flakes.

The particularly preferred effect pigments are mica flakes which are covered with TiO₂, Fe₂O₃, Fe₃O₄, TiO₂/Fe₂O₃ or TiO₂/Fe₃O₄. The admixing of natural or nature-identical dyes, organic or inorganic coloured pigments or colouring natural fruit and plant extracts which are approved for the feed products sector enables the colour effect of the effect pigments in the product to be influenced and at the same time enables novel iridescent colour effects to be achieved.

Besides the effect pigments, all natural or nature-identical dyes which are known to the person skilled in the art can be admixed with the feed product or animal medicament as further colouring component. In particular, mention should be made here of E 101, E 104, E 110, E 124, E 131, E 132, E 140, E 141, E 151, E 160a. Furthermore, it is also possible to admix other coloured pigments with the flake-form effect pigments, such as, for example, E 171, E 172, E 153.

Besides the effect pigments, the proportion of dyes, based on the feed product, is preferably in the range from 0.5 to 25% by weight. The dyes employed can likewise be fruit and plant extracts, such as, for example, carrot juice, beetroot juice, elderberry juice, hibiscus juice, paprika extract, aronia extract.

The effect pigments which are approved for the feed products sector and for animal medicaments are commercially available, for example under the brand Candurin® from Merck KGaA.

The total concentration of all pigments in the product to be pigmented should not exceed 50% by weight, based on the product. It is generally dependent on the specific application.

Various active-ingredient additives, such as, for example, vitamins, enzymes, trace elements, proteins, carbohydrates, essential fats and/or minerals, can also be added to the feed products and animal medicaments, where the total amount of active ingredients, based on the feed product or animal medicament, should not exceed 25% by weight. The amount of active ingredients or active-ingredient mixtures is preferably 0.01-20% by weight.

The products are coloured by adding the effect pigment or effect-pigment mixture to the product to be coloured, alone or in combination with further pigments or colorants, directly or in the presence of water and/or an organic solvent in the desired mixing ratios, simultaneously or successively, during or after the manufacture thereof, before or after shaping (for example during extrusion, pelleting, expansion, granulation, etc.). Admixing of the effect pigments with pulverulent or loose feed product mixtures is likewise possible.

The effect pigments can also be applied to the surface in order to colour the feed products or animal medicaments after shaping. In this case, the effect pigment is generally mixed with an application medium and subsequently applied to the product using suitable application and spray devices. The application or coating composition then ensures the corresponding adhesion of the pigments to the product surface. The latter is then coloured correspondingly.

On incorporation into the product matrix itself, the amount of effect pigments used is preferably 0.5-40% by weight, in particular 1-30% by weight. In the case of surface colouring of feed products or animal medicament preparations, the amount used in the colouring or coating solution used is in the range 0.1-25% by weight, in particular 1-15% by weight. On use of the effect pigments in pulverulent products, the use range is 0.05-50% by weight, in particular 2-10% by weight.

The coating solutions preferably comprise water or organic solvents, such as, for example, ethanol or isopropanol. The film former employed in the coating solutions is preferably a cellulose derivative, such as, for example, hydroxypropylmethylcellulose. Particular preference is given to application solutions comprising cellulose derivatives which, instead of water, comprise 5-80% by weight of a suitable organic solvent.

Compared with aqueous coating solutions, the alcoholic or alcoholic-aqueous, cellulose-containing application solutions have significant applicational advantages:
use of cooler drying air during the spray application
colouring of heat-sensitive products, such as, for example, vitamin-containing feed products, with effect pigments is very readily possible.

Further products that may be mentioned which are suitable for colouring are, in particular, coatings on all types of feed products, in particular pigmented sugar and shellac coatings (alcoholic and aqueous), coatings with oils, fats, starches and waxes, with gum arabic, with cellulose derivatives (for example HPMC=hydroxypropylmethylcellulose), with starch and albumin derivatives, carrageenan and other substances known to the person skilled in the art which are suitable for coating. The effect pigment is generally mixed with the application medium here and subsequently applied to the feed product using suitable application and spray devices, or by hand. The application or coating composition then ensures the corresponding adhesion of the pigments to the feed product or animal medicament surface. The latter is then coloured correspondingly.

The application and coating solutions preferably comprise 0.1-20% by weight, in particular 2-15% by weight, of effect pigments.

Preferred dry powder mixtures for coatings comprise a cellulose derivative, such as, for example, hydroxypropylmethylcellulose, sodium carboxymethylcellulose, a release agent, such as, for example, lecithin or stearic acid, a lustre enhancer, such as, for example, maltodextrin and/or dextrose, and an effect pigment or effect-pigment mixture. Dry powder mixtures of this type preferably comprise the effect pigment in amounts of 0.01-50% by weight, in particular 0.5-40% by weight, based on the powder mixture. If necessary, dyes, flavours, vitamins, sweeteners, etc., can be added to these dry powder mixtures.

In the case of coated feed products and animal medicaments, the combination of the effect pigments with aroma substances (powder or liquid aromas), acids and/or with sweeteners, such as, for example, aspartame, is possible in order to emphasise the optical effect also in taste terms.

The invention thus relates to all formulations from the feed products and animal medicaments sector comprising one or more effect pigments, alone or in combination with further pigments/pigment mixtures or dyes (natural or nature-identical) as colorants.

The following examples are intended to explain the invention, but without limiting it.

### USE EXAMPLES

### Example 1

### Pelleting of Pig Fattening Feed

Feed product mixture: finisher feed for pigs initial bulk density 0.61 kg/dm³, initial moisture content 8.62%

| Press: | Kahl 14-175 laboratory press |
|---|---|
| Number of rollers: | 2 |
| Rotational speed: | 112 rpm |
| Roller speed: | 0.73 m/s |
| Die: | diameter 175*27*3.0 mm |
| Pitch: | 6.0 |
| Pressing ratio | 1:9.0 |

Preparation of the Pelleting Mixture in the Turbomixer Before Pelleting:

| | |
|---|---|
| 10% of Candurin ® Silver Sparkle* | Merck KGaA |
| 3% of water | |
| 87% of feed product mixture | |
| 100% of pelleting mixture | |

(*Candurin ® Silver Sparkle: TiO₂— coated mica pigment having a particle size of 10-150 μm, Merck KGaA)

[symbol] slight silvery sparkle effect evident on the pellet surfaces

### Example 2

### Colouring of Fish Food Extrudates

Starting material to be coloured:

Commercially available extruded fish food, particle size 4*4 mm, initial bulk density: 0.46 kg/dm³, initial moisture content: 4.2%

| Coating System: | Kahl Totospray | |
|---|---|---|
| Fish food throughput: | 900 | kg/h |
| Application suspension throughput: | 9.0 | kg/h |
| Spray rate: | 1.0% | |
| Atomiser rotational speed: | 6000 | rpm |
| Spray duration: | 3 | min |

Preparation of the Application Suspension:

| | |
|---|---|
| 91% of water | |
| 5% of Sepifilm 050 | Seppic SA |
| 4% of Candurin ® Silver Sparkle | Merck KGaA |
| 100% of application suspension | |

Suspension is prepared about 1 hour before commencement of the experiment.

Sepifilm 050 is a film-formation system based on hydroxypropylmethylcellulose.

[symbol] sparkly silver colour effect on the extrudate surface

### Example 3

### Colouring of Fish Food Extrudates

Experiment analogous to Experiment 2 with increased proportion of effect pigment.

Application Suspension:

| | |
|---|---|
| 87% of water | |
| 5% of Sepifilm 050 | Seppic SA |
| 8% of Candurin ® Silver Sparkle | Merck KGaA |
| 100% of application suspension | |

[symbol] increased sparkly silver colour effect on the extrudate surface

### Example 4

### Colouring of Fish Food Extrudates

Experiment analogous to Experiment 2 with further increased proportion of effect pigment

Application Suspension:

| | |
|---|---|
| 83% of water | |
| 5% of Sepifilm 050 | Seppic SA |
| 12% of Candurin ® Silver Sparkle | Merck KGaA |
| 100% of application suspension | |

[symbol] very strong sparkly silver colour effect on the extrudate surface

### Example 5

### Colouring of Fish Food Extrudates

Experiment analogous to Experiment 3 with effect pigments based on iron oxide/mica.

Application Suspension:

| | |
|---|---|
| 87% of water | |
| 5% of Sepifilm 050 | Seppic SA |
| 8% of Candurin ® Red Sparkle* | Merck KGaA |
| 100% of application suspension | |

(*Candurin ® Red Sparkle: Fe₂O₃— coated mica flakes having a particle size of 10-150 μm, Merck KGaA)

[symbol] attractive sparkly red colour effect on the extrudate surface

### Example 6

### Colouring of Pet Food Extrudates (Hamster Food)

Colouring is carried out by spraying on the application suspension by means of a spray gun in an open coating pan.

| | |
|---|---|
| Spray gun: | SATAminijet NR |
| Nozzle diameter: | 1.0 mm |
| Compressed air: | 3 bar |
| Coating pan: | Trybuhl 5 l laboratory pan |

Coating line with temperature-controllable drying air supply

| | |
|---|---|
| Temperature of the drying air: | 45° C. |
| Amount of application suspension: | 6% (=60 g/kg of extrudates) |

Application Suspension:

| | |
|---|---|
| 90% of water | |
| 5% of Sepifilm 050 | Seppic SA |
| 5% of Candurin ® Gold Lustre* | Merck KGaA |
| 100% of application suspension | |

(*Candurin ® Gold Lustre: TiO₂— and Fe₂O₃— coated mica flakes having a particle size of 10-60 μm, Merck KGaA)

[symbol] very attractive uniform, golden lustre effect on the extrudate surface

### Example 7

### Colouring of Bird Seed

Colouring is carried out by spraying on the application suspension by means of a spray gun in an open coating pan.

| | |
|---|---|
| Spray gun: | SATAminijet NR |
| Nozzle diameter: | 1.0 mm |
| Compressed air: | 3 bar |
| Coating pan: | Trybuhl 5 l laboratory pan |

Coating line with temperature-controllable drying air supply

| | |
|---|---|
| Temperature of the drying air: | 25° C. |
| Amount of application suspension: | 3% (=30 g/kg of extrudates) |

Application Suspension:

| | |
|---|---|
| 20% of Capol 425 | Kaul GmbH |
| 77% of ethanol (96%) | Merck KGaA |
| 3% of Candurin ® Gold Shimmer* | Merck KGaA |
| 100% of application suspension | |

(*Candurin ® Gold Shimmer interference effect pigment; TiO₂-coated mica flakes having a particle size of 10-60 μm from Merck KGaA)
Capol 425: ethanolic shellac solution

[symbol] very attractive, uniform, golden mother-of-pearl lustre effect on the seed surface

### Example 8

### Colouring of Pet Food Extrudates (Hamster Food)

Colouring is carried out by hand application of a Candurin® suspension in an open coating pan.

Application Suspension:

| | |
|---|---|
| 40% of Capol 425 | Kaul GmbH |
| 50% of ethanol (96%) | Merck KGaA |
| 10% of Candurin ® Silver Sparkle* | Merck KGaA |
| 100% of application suspension | |

The ready-mixed Candurin ® suspension is applied by hand to the pellets in one or more steps. After the desired distribution of the pigments on the product surface, the coating pan is stopped and dried using cold or warm air. In order to prevent the pigments from caking together, the coating pan is periodically switched on briefly.
(*Candurin ® Silver Sparkle: TiO₂-coated mica pigment having a particle size of 10-150 μm, Merck KGaA)
Capol 425: ethanolic shellac solution

[symbol] sparkly silver, mother-of-pearl lustre effect on the pellet surface

### Example 9

### Colouring of Fish Food (Expanded Products)

Colouring is carried out by hand application of a Candurin® suspension in an open coating pan.

Application Suspension:

| | |
|---|---|
| 80% of Quick Polish | A. Wolf GmbH |
| 10% of ethanol (96%) | Merck KGaA |
| 10% of Candurin ® Brown Amber* | Merck KGaA |
| 100% of application suspension | |

The ready-mixed Candurin ® suspension is applied by hand to the pellets in one or more steps. After the desired distribution of the pigments on the product surface, the coating pan is stopped and dried using cold or warm air. In order to prevent the pigments from caking together, the coating pan is periodically switched on briefly.
(*Candurin ® Brown Amber: Fe₂O₃-coated mica flakes having a particle size of 10-60 μm, Merck KGaA)
Quick Polish: gum arabic solution

[symbol] sparkly bronze, mother-of-pearl lustre effect on the pellet surface

### Example 10

### Colouring of Fish Food (Expanded Products)

Colouring is carried out by hand application of a Candurin® suspension in an open coating pan.

Application Suspension:

| | |
|---|---|
| 80% of Quick Oil | A. Wolf GmbH |
| 10% of ethanol (96%) | Merck KGaA |
| 10% of Candurin ® Gold Lustre* | Merck KGaA |
| 100% of application suspension | |

The ready-mixed Candurin ® oil suspension is applied by hand to the pellets in one or more steps. After the desired distribution of the pigments on the product surface, the coating pan is stopped and dried using cold or warm air. In order to prevent the pigments from caking together, the coating pan is periodically switched on briefly.
(*Candurin ® Gold Lustre: TiO₂— and Fe₂O₃-coated mica flakes having a particle size of 10-60 μm, Merck KGaA)
Quick Oil: oil- and wax-based polish

[symbol] sparkly gold, mother-of-pearl lustre effect on the pellet surface

### Example 11

### Colouring of Pet Food Extrudates (Hamster Food)

Colouring is carried out by spraying on the application suspension by means of a spray gun in an open coating pan.

| | |
|---|---|
| Spray gun: | SATAminijet NR |
| Nozzle diameter: | 1.0 mm |
| Compressed air: | 3 bar |
| Coating pan: | Trybuhl 5 I laboratory pan |

Coating line with temperature-controllable drying air supply

| | |
|---|---|
| Temperature of the drying air: | 35° C. |
| Amount of application suspension: | 80% (=80 g/kg of extrudates) |

Application Suspension:

| | |
|---|---|
| 60% of water | |
| 30% of ethanol (96%) | Merck KGaA |
| 5% of Aquapolish | Biogrund GmbH |
| 5% of Candurin ® Timiron Spledid Blue* | Merck KGaA |
| 100% of application suspension | |

Suspension is prepared about 1 hour before commencement of the experiment.
Aquapolish is a film-formation system based on hydroxypropylmethylcellulose.
(*Candurin ® Timiron Splendid Blue: TiO₂—, SiO₂— and Fe₂O₃-coated multi-layered pigment based on mica having a particle size of 10-60 μm, Merck KGaA)
Aquapolish: film

[symbol] very attractive uniform, blue lustre effect on the extrudate surface

### Example 12

### Colouring of Fish Food Expandates

Colouring is carried out by spraying on the application suspension by means of a spray gun in an open coating pan

| | |
|---|---|
| Spray gun: | SATAminijet NR |
| Nozzle diameter: | 1.0 mm |
| Compressed air: | 3 bar |
| Coating pan: | Trybuhl 5 I laboratory pan |

Coating line with temperature-controllable drying air supply

| | |
|---|---|
| Temperature of the drying air: | 45° C. |
| Amount of application suspension: | 5% (=50 g/kg of extrudates) |

Application Suspension:

| | |
|---|---|
| 91% of water | |
| 5% of Opadry dry clear | Colorcon GmbH |
| 4% of Ronastar ™ Noble Sparks* | Merck KGaA |
| 100% of application suspension | |

(*Ronastar ™ Noble Sparks: SiO₂— and TiO₂-coated effect pigment based on aluminium borosilicate glass having a particle size of 20-200 μm, Merck KGaA)

Suspension is prepared about 1 hour before commencement of the experiment.

Opadry II clear is a film-formation system based on hydroxypropylmethylcellulose.

[symbol] very attractive sparkly and uniform, silver lustre effect on the product surface

### Example 13

### Colouring of Feed Pellets for Salt-Water Crabs

Colouring is carried out by hand application in the rotating coating pan:

Application Composition Consists of:

| | |
|---|---|
| 10% of Candurin ® Silver Sparkle* | Merck KGaA |
| 90% of Couva 300H | Loders Croklaan B.V. |

Couva 300H is a vegetable fat. This is warmed to about 40-45° C. The Candurin ® pigments are subsequently added.
Application rate: 1-15%, depending on the desired colour effect
(*Candurin ® Silver Sparkle: TiO₂-coated mica pigment having a particle size of 10-150 μm, Merck KGaA)
Couva 300H: plant-based edible fat

### Example 14

### Colouring of Pellets for Salt-Water Crabs

Colouring is carried out by hand application in the rotating coating pan:

Application Composition Consists of:

| | |
|---|---|
| 5% of Candurin ® Red Sparkle | Merck KGaA |
| 90% of Couva 300H | Loders Croklaan B.V. |

Couva 300H is a vegetable fat. This is warmed to about 40-45° C. The Candurin ® pigments are subsequently added.
Application rate: 1-15%, depending on the desired colour effect
(*Candurin ® Red Sparkle: Fe₂O₃-coated mica flake having a particle size of 10-150 μm, Merck KGaA)
Couva 300H: plant-based edible fat

1. Use of effect pigments based on flake-form substrates for colouring feed products and animal medicaments.

2. Use of effect pigments for colouring feed products and animal medicaments according to claim 1, characterised in that the substrate is a natural and/or synthetic mica flake, talc, kaolin, flake-form iron or aluminium oxide, glass flake, SiO₂ flake, TiO₂ flake, flake-form mixed oxide or graphite flake.

3. Use of effect pigments for colouring feed products and animal medicaments according to claim 1, characterised in that the substrate has one or more metal-oxide layers.

4. Use of effect pigments for colouring feed products and animal medicaments according to claim 1, characterised in that the substrates are coated with TiO₂, Fe₂O₃ and/or Fe₃O₄.

5. Use of effect pigments according to claim 1, characterised in that the multilayered pigments have alternating high- and low-refractive-index layers.

6. Use of effect pigments according to claim 1, characterised in that the effect pigment has the following layer structure:
substrate + TiO₂
substrate + Fe₂O₃
substrate + Fe₃O₄
substrate + TiO₂ + SiO₂ + TiO₂
substrate + TiO₂ + SiO₂ + Fe₂O₃
substrate + TiO₂ + SiO₂ + Fe₃O₄
substrate + TiO₂ + Al₂O₃ + TiO₂
substrate + TiO₂ + Al₂O₃ + Fe₂O₃
substrate + TiO₂ + Al₂O₃ + Fe₂O₃
substrate + Fe₂O₃ + SiO₂ + TiO₂
substrate + Fe₃O₄ + SiO₂ + TiO₂
substrate + Fe₂O₃ + Al₂O₃ + TiO₂
substrate + Fe₃O₄ + Al₂O₃ + TiO₂
substrate + SiO₂ + TiO₂
substrate + SiO₂ + Fe₂O₃
substrate + SiO₂ + Fe₃O₄

7. Use of effect pigments according to claim 1, characterised in that the proportion of effect pigment in the feed product or in the animal medicament is 0.01 to 50% by weight.

8. Use of effect pigments according to claim 1, characterised in that effect pigments are employed in combination with natural or nature-identical dyes, coloured pigments or natural colouring plant or fruit extracts.

9. Use of effect pigments according to claim 1, characterised in that the feed product and the animal medicament preparation is provided with a coating of cellulose derivatives, shellac, oils, waxes, gum arabic, cellulose types, polymethacrylates, based on starch or albumin, fats and fat derivatives or sugar frosting comprising effect pigments and optionally further pigments and/or colorants.

10. Process for the manufacture of feed products or animal medicaments coloured using effect pigments, characterised in that the effect pigment or effect pigment mixture is added to the product to be coloured or applied to the surface of the feed product or to the animal medicament preparation, alone or in combination with further pigments or colorants, directly or in the presence of water and/or of an organic solvent in the desired mixing ratios, simultaneously or successively, during or after the manufacture thereof, before or after shaping.

11. Feed products and animal medicaments comprising effect pigments based on flake-form substrates as colorants.
